(19) United States
(12) Patent Application Publication
(10) Pub. No.: US 2018/0325625 A1
(43) Pub. Date: Nov. 15, 2018

(54) MOUTHGUARD SYSTEM

(71) Applicant: P & D Mouthguards, Inc., Jacksonville, FL (US)

(72) Inventor: Anthony R. Petrone, Neptune Beach, FL (US)

(21) Appl. No.: 15/977,589

(22) Filed: May 11, 2018

Related U.S. Application Data
(60) Provisional application No. 62/504,756, filed on May 11, 2017.

(51) Int. Cl. A61C 7/08; A61F 5/56; A63B 71/08

(52) U.S. Cl. CPC A61C 7/08; A61C 7/36; A63B 71/085; A61F 5/566

(57) ABSTRACT

A mouthguard system includes an upper mouthguard assembly configured to be positionable about a maxilla of a user; a lower mouthguard assembly configured to be positionable about a mandible of the user; and one or more stabilizer systems configured to align the upper mouthguard assembly with the lower mouthguard assembly.

### RELATED APPLICATION(S)

This application claims the benefit of U.S. Provisional Application No. 62/504,756, filed on 11 May 2017, the entire contents of which are incorporated herein by reference.

### TECHNICAL FIELD

This disclosure relates to mouthguard systems and, more particularly, to mouthguard systems that include magnets.

### BACKGROUND

Mouthguards may be used as protective devices for the mouth that cover the teeth and gums to prevent and/or reduce injury to the teeth, arches, lips and gums. A mouthguard may be used to prevent injury in contact sports, as a treatment for bruxism or TMD, or as part of certain dental procedures (e.g., tooth bleaching or sleep apnea).

Unfortunately, current mouthguards may not be optimized to perform their intended function. For example, current mouthguards may not be designed to optimize alignment of the jaw with respect to the skull of the user. Further, such current mouthguards may not be configured to properly position the jaw with respect to the skull of the user.

### SUMMARY OF DISCLOSURE

In one implementation, a mouthguard system includes an upper mouthguard assembly configured to be positionable about a maxilla of a user; a lower mouthguard assembly configured to be positionable about a mandible of the user; and one or more stabilizer systems configured to align the upper mouthguard assembly with the lower mouthguard assembly.

One or more of the following features may be included. The upper mouthguard assembly may include an inner layer positionable proximate one or more teeth of the maxilla of the user. The inner layer of the upper mouthguard assembly may be configured to be compliant so that it may mold to the shape of the one or more teeth of the maxilla of the user. The upper mouthguard assembly may further include an outer layer positioned on top of the inner layer of the upper mouthguard assembly. The outer layer of the upper mouthguard assembly may be configured to be resilient and protect the inner layer of the upper mouthguard assembly and/or the one or more teeth of the maxilla of the user. The lower mouthguard assembly may include an inner layer positionable proximate one or more teeth of the mandible of the user. The inner layer of the lower mouthguard assembly may be configured to be compliant so that it will mold to the shape of the one or more teeth of the mandible of the user. The lower mouthguard assembly may further include an outer layer positioned on top of the inner layer of the lower mouthguard assembly. The outer layer of the lower mouthguard assembly may be configured to be resilient and protect the inner layer of the lower mouthguard assembly and/or the one or more teeth of the mandible of the user. The one or more stabilizer systems may include a displacement magnetic system configured to move the mandible forward with respect to the maxilla of the user. The displacement magnetic system may include a first magnetic element coupled to the upper mouthguard assembly and a second magnetic element coupled to the lower mouthguard assembly. The first magnetic element and the second magnetic element may be configured to generate an attractive magnetic force. The one or more stabilizer systems may include a cushioning magnetic system configured to move the mandible downward with respect to the maxilla of the user. The cushioning magnetic system may include a first magnetic element coupled to the upper mouthguard assembly and a second magnetic element coupled to the lower mouthguard assembly. The first magnetic element and the second magnetic element may be configured to generate a repelling magnetic force.

In another implementation, a mouthguard system includes an upper mouthguard assembly configured to be positionable about a maxilla of a user; a lower mouthguard assembly configured to be positionable about a mandible of the user; and one or more stabilizer systems configured to align the upper mouthguard assembly with the lower mouthguard assembly. The one or more stabilizer systems includes: a displacement magnetic system configured to move the mandible forward with respect to the maxilla of the user and including: a first magnetic element coupled to the upper mouthguard assembly, and a second magnetic element coupled to the lower mouthguard assembly, wherein the first magnetic element and the second magnetic element are configured to generate an attractive magnetic force.

One or more of the following features may be included. The one or more stabilizer systems may include a cushioning magnetic system configured to move the mandible downward with respect to the maxilla of the user. The cushioning magnetic system may include a first magnetic element coupled to the upper mouthguard assembly and a second magnetic element coupled to the lower mouthguard assembly.

In another implementation, a mouthguard system includes: an upper mouthguard assembly configured to be positionable about a maxilla of a user; a lower mouthguard assembly configured to be positionable about a mandible of the user; and one or more stabilizer systems configured to align the upper mouthguard assembly with the lower mouthguard assembly. The one or more stabilizer systems includes a cushioning magnetic system configured to move the mandible downward with respect to the maxilla of the user and including: a first magnetic element coupled to the upper mouthguard assembly, and a second magnetic element coupled to the lower mouthguard assembly, wherein the first and second magnetic elements are configured to generate a repelling magnetic force.

One or more of the following features may be included. The one or more stabilizer systems may include a displacement magnetic system configured to move the mandible forward with respect to the maxilla of the user.

The details of one or more implementations are set forth in the accompanying drawings and the description below. Other features and advantages will become apparent from the description, the drawings, and the claims.

### BRIEF DESCRIPTION OF THE DRAWINGS

FIG. 1 is a perspective view of a mouthguard system accordingly to an implementation of this disclosure;

FIG. 2 is a side view of a human skull; and

FIG. 3 is a cross sectional view of the mouthguard system of FIG. 1.

Like reference symbols in the various drawings indicate like elements.

### DETAILED DESCRIPTION OF THE PREFERRED EMBODIMENTS

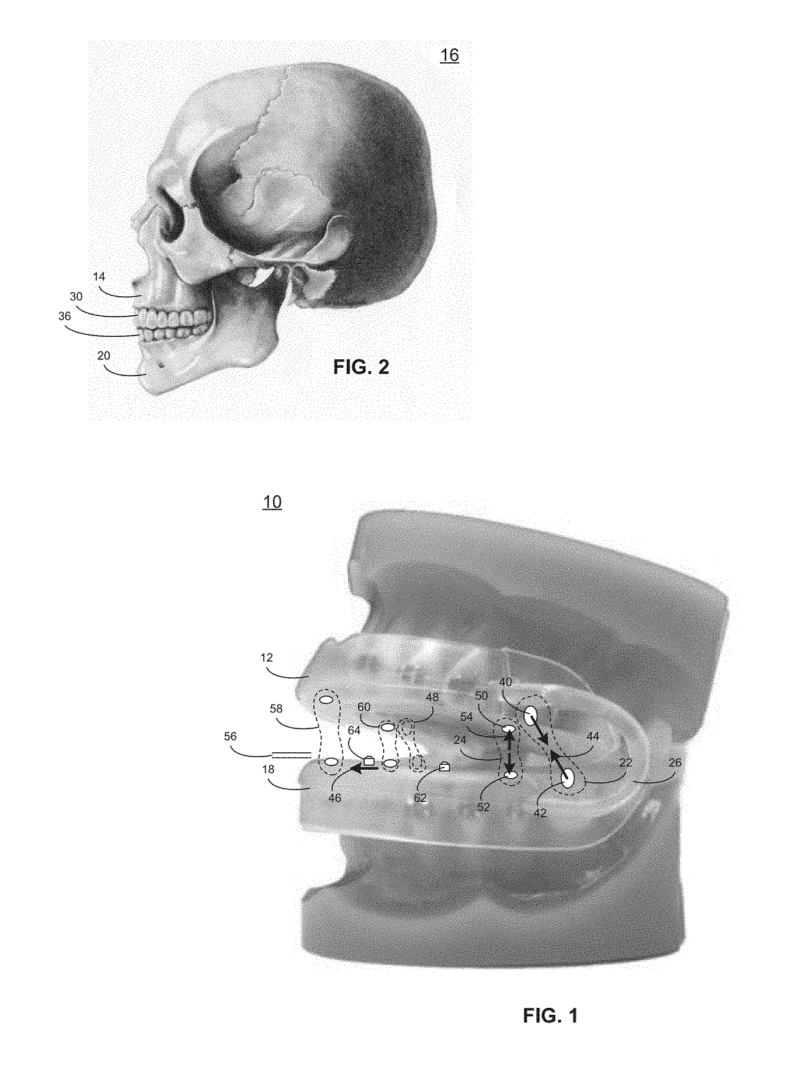

Referring to FIGS. 1-2, there is shown mouthguard system 10. Mouthguard system 10 may include upper mouthguard assembly 12 configured to be positionable about maxilla 14 of user 16. Mouthguard system 10 may also include lower mouthguard assembly 18 configured to be positionable about mandible 20 of user 16.

Mouthguard system 10 may include one or more stabilizer systems (e.g., stabilizer systems 22, 24) configured to align upper mouthguard assembly 12 with lower mouthguard assembly 18. Depending upon the manner in which mouthguard system 10 is configured, upper mouthguard assembly 12 and lower mouthguard assembly 18 may be coupled together. For example, upper mouthguard assembly 12 and lower mouthguard assembly 18 may be separate and discrete components. Alternatively, upper mouthguard assembly 12 and lower mouthguard assembly 18 may be coupled together (via e.g., coupler 26) so that upper mouthguard assembly 12 and lower mouthguard assembly 18 e.g., do not get separated.

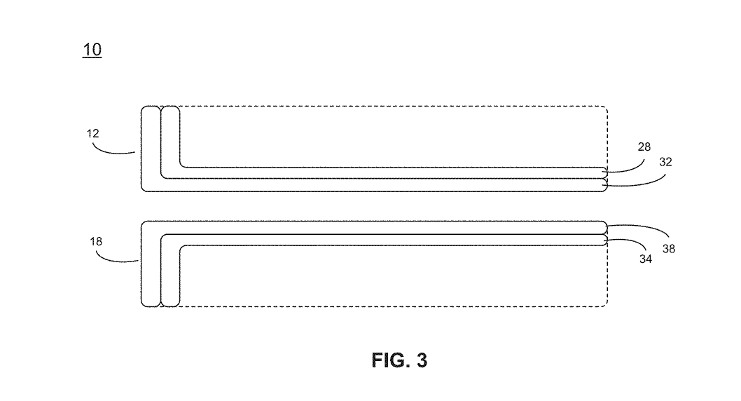

Referring also to FIG. 3, upper mouthguard assembly 12 may include inner layer 28 positionable proximate one or more teeth (e.g., teeth 30) of maxilla 14 of user 16. Inner layer 28 of upper mouthguard assembly 12 may be configured to be compliant so that it may mold to the shape of the one or more teeth (e.g., teeth 30) of maxilla 14 of user 16. For example, inner layer 28 may be constructed of ethylene-vinyl acetate (EVA), also known as poly-ethylene-vinyl acetate (PEVA).

As is known in the art, ethylene-vinyl acetate (EVA) is the copolymer of ethylene and vinyl acetate. The weight percentage of vinyl acetate may vary from 10% to 40%, with the remainder being ethylene. Broadly speaking, there may be three different types of ethylene-vinyl acetate (EVA), which may differ in the vinyl acetate (VA) content and the manner in which the materials are used. Ethylene-vinyl acetate (EVA) copolymer that is based upon a low proportion of VA (approximately up to 4%) may be referred to as vinyl acetate modified polyethylene (which is a copolymer) that may be processed as a thermoplastics material (like low density polyethylene) and may have some of the properties of a low density polyethylene but with increased gloss, softness and flexibility. As is understood, ethylene-vinyl acetate (EVA) is merely intended to be an example of the type of material from which inner layer 28 of upper mouthguard assembly 12 may be constructed. Accordingly, this is for illustrative purposes only, is not intended to be exhaustive, and is in no way intended to be a limitation concerning the scope of this disclosure.

Upper mouthguard assembly 12 may further include outer layer 32 positioned on top of inner layer 28 of upper mouthguard assembly 12. Outer layer 32 may be positioned on top of inner layer 28 of upper mouthguard assembly 12 via e.g., a vacuum forming process. Outer layer 32 of upper mouthguard assembly 12 may be configured to be resilient and protect inner layer 28 of upper mouthguard assembly 12 and/or the one or more teeth (e.g., teeth 30) of maxilla 14 of user 16. For example, outer layer 32 may also be constructed of ethylene-vinyl acetate (EVA). However, the specific formulation of the ethylene-vinyl acetate (EVA) copolymer used to construct outer layer 32 may be adjusted to allow outer layer 32 to provide a higher level of resilience when compared to inner layer 28. As is understood, ethylene-vinyl acetate (EVA) is merely intended to be an example of the type of material from which outer layer 32 of upper mouthguard assembly 12 may be constructed. Accordingly, this is for illustrative purposes only, is not intended to be exhaustive, and is in no way intended to be a limitation concerning the scope of this disclosure.

Lower mouthguard assembly 18 may include inner layer 34 positionable proximate one or more teeth (e.g., teeth 36) of mandible 20 of user 16. Inner layer 34 of lower mouthguard assembly 18 may be configured to be compliant so that it may mold to the shape of the one or more teeth (e.g., teeth 36) of mandible 20 of user 16. In a fashion similar to that of inner layer 28 of upper mouthguard assembly 12, inner layer 34 of lower mouthguard assembly 18 may be constructed of ethylene-vinyl acetate (EVA) to allow inner layer 34 to have a high level of softness and flexibility. As is understood, ethylene-vinyl acetate (EVA) is merely intended to be an example of the type of material from which inner layer 34 of lower mouthguard assembly 18 may be constructed. Accordingly, this is for illustrative purposes only, is not intended to be exhaustive, and is in no way intended to be a limitation concerning the scope of this disclosure.

Lower mouthguard assembly 18 may further include outer layer 38 positioned on top of inner layer 34 of lower mouthguard assembly 18. Outer layer 38 may be positioned on top of inner layer 34 of lower mouthguard assembly 18 via e.g., a vacuum forming process. As with outer layer 32 of upper mouthguard assembly 12, outer layer 38 of lower mouthguard assembly 18 may be configured to be resilient and protect inner layer 34 of lower mouthguard assembly 18 and/or the one or more teeth (e.g., teeth 36) of mandible 20 of user 16. For example, outer layer 38 may also be constructed of ethylene-vinyl acetate (EVA). However, the specific formulation of the ethylene-vinyl acetate (EVA) copolymer used to construct outer layer 38 may be adjusted to allow outer layer 38 to provide a higher level of resilience when compared to inner layer 34. As is understood, ethylene-vinyl acetate (EVA) is merely intended to be an example of the type of material from which outer layer 38 of lower mouthguard assembly 18 may be constructed. Accordingly, this is for illustrative purposes only, is not intended to be exhaustive, and is in no way intended to be a limitation concerning the scope of this disclosure.

As discussed above, mouthguard system 10 may include one or more stabilizer systems (e.g., stabilizer systems 22, 24) configured to align upper mouthguard assembly 12 with lower mouthguard assembly 18. While the following discussion concerns stabilizer systems 22, 24 being magnetic in nature, this is for illustrative purposes only and is not intended to be a limitation of this disclosure, as other configurations are possible and are considered to be within the scope of this disclosure. For example, stabilizer systems 22, 24 may be mechanical in nature and may use mechanical systems (e.g., rubber bands, elastic linkages, springs, elastic cushions, elastic pads, etc.) to provide the aligning forces.

The one or more stabilizer systems (e.g., stabilizer systems 22, 24) may include a displacement magnetic system (e.g., displacement magnetic system 22) configured to move mandible 20 of user 16 forward with respect to maxilla 14 of user 16.

Displacement magnetic system 22 may include first magnetic element 40 coupled to upper mouthguard assembly 12 and second magnetic element 42 coupled to lower mouthguard assembly 18. First magnetic element 40 and second magnetic element 42 may be configured to generate an attractive magnetic force (e.g., attractive force 44). As is known in the art and through the use of attractive magnetic force 44, by moving mandible 20 of user 16 forward (in the direction of arrow 46) with respect to maxilla 14 of user 16, the airway (not shown) of user 16 may be opened up, thus allowing for a higher level of respiratory function and a higher level of blood oxygenation.

Mouthguard system 10 may include a plurality of displacement magnetic systems (e.g., displacement magnetic systems 22, 48), wherein the discrete magnetic elements of these displacement magnetic systems (e.g., displacement magnetic systems 22, 48) may be positioned between inner layer 28 and outer layer 32 of upper mouthguard assembly 12 and/or inner layer 34 and outer layer 38 of lower mouthguard assembly 18.

Further, the one or more stabilizer systems (e.g., stabilizer systems 22, 24) may include a cushioning magnetic system (e.g., cushioning magnetic system 24) configured to move mandible 20 of user 16 downward with respect to maxilla 14 of user 16.

Cushioning magnetic system 24 may include first magnetic element 50 coupled to upper mouthguard assembly 12 and second magnetic element 52 coupled to lower mouthguard assembly 18. First magnetic element 50 and second magnetic element 52 may be configured to generate a repelling magnetic force (e.g., repelling force 54) to maintain a separation space (e.g., separation space 56) between upper mouthguard assembly 12 and lower mouthguard assembly 18. Mouthguard system 10 may include a plurality of cushioning magnetic systems (e.g., cushioning magnetic system 24, 58, 60), wherein the discrete magnetic elements of these cushioning magnetic systems (e.g., cushioning magnetic system 24, 58, 60) may be positioned between inner layer 28 and outer layer 32 of upper mouthguard assembly 12 and/or inner layer 34 and outer layer 38 of lower mouthguard assembly 18.

Mouthguard system 10 may include one or more friction reducing assemblies (e.g., roller assemblies 62, 64) configured to reduce the friction between upper mouthguard assembly 12 and lower mouthguard assembly 18. For example, roller assemblies 52, 54 may be configured to reduce friction between upper mouthguard assembly 12 and lower mouthguard assembly 18, thus allowing for lateral displacement of mandible 20 with respect to maxilla 14 of user 16 (in the direction of arrow 56).

### General:

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

A number of implementations have been described. Having thus described the disclosure of the present application in detail and by reference to embodiments thereof, it will be apparent that modifications and variations are possible without departing from the scope of the disclosure defined in the appended claims.

What is claimed is:

1. A mouthguard system comprising:
an upper mouthguard assembly configured to be positionable about a maxilla of a user;
a lower mouthguard assembly configured to be positionable about a mandible of the user; and
one or more stabilizer systems configured to align the upper mouthguard assembly with the lower mouthguard assembly.

2. The mouthguard system of claim 1 wherein the upper mouthguard assembly includes:
an inner layer positionable proximate one or more teeth of the maxilla of the user.

3. The mouthguard system of claim 2 wherein the inner layer of the upper mouthguard assembly is configured to be compliant so that it may mold to the shape of the one or more teeth of the maxilla of the user.

4. The mouthguard system of claim 2 wherein the upper mouthguard assembly further includes:
an outer layer positioned on top of the inner layer of the upper mouthguard assembly.

5. The mouthguard system of claim 4 wherein the outer layer of the upper mouthguard assembly is configured to be resilient and protect the inner layer of the upper mouthguard assembly and/or the one or more teeth of the maxilla of the user.

6. The mouthguard system of claim 1 wherein the lower mouthguard assembly includes:
an inner layer positionable proximate one or more teeth of the mandible of the user.

7. The mouthguard system of claim 6 wherein the inner layer of the lower mouthguard assembly is configured to be compliant so that it will mold to the shape of the one or more teeth of the mandible of the user.

8. The mouthguard system of claim 6 wherein the lower mouthguard assembly further includes:
an outer layer positioned on top of the inner layer of the lower mouthguard assembly.

9. The mouthguard system of claim 8 wherein the outer layer of the lower mouthguard assembly is configured to be resilient and protect the inner layer of the lower mouthguard assembly and/or the one or more teeth of the mandible of the user.

10. The mouthguard system of claim 1 wherein the one or more stabilizer systems includes:
a displacement magnetic system configured to move the mandible forward with respect to the maxilla of the user.

11. The mouthguard system of claim 10 wherein the displacement magnetic system includes:
a first magnetic element coupled to the upper mouthguard assembly; and
a second magnetic element coupled to the lower mouthguard assembly.

12. The mouthguard system of claim 11 wherein the first magnetic element and the second magnetic element are configured to generate an attractive magnetic force.

13. The mouthguard system of claim 1 wherein the one or more stabilizer systems includes:
a cushioning magnetic system configured to move the mandible downward with respect to the maxilla of the user.

14. The mouthguard system of claim 13 wherein the cushioning magnetic system includes:
a first magnetic element coupled to the upper mouthguard assembly; and
a second magnetic element coupled to the lower mouthguard assembly.

15. The mouthguard system of claim 14 wherein the first magnetic element and the second magnetic element are configured to generate a repelling magnetic force.

16. A mouthguard system comprising:
an upper mouthguard assembly configured to be positionable about a maxilla of a user;
a lower mouthguard assembly configured to be positionable about a mandible of the user; and
one or more stabilizer systems configured to align the upper mouthguard assembly with the lower mouthguard assembly, wherein the one or more stabilizer systems includes:
a displacement magnetic system configured to move the mandible forward with respect to the maxilla of the user and including:
a first magnetic element coupled to the upper mouthguard assembly, and
a second magnetic element coupled to the lower mouthguard assembly,
wherein the first magnetic element and the second magnetic element are configured to generate an attractive magnetic force.

17. The mouthguard system of claim 16 wherein the one or more stabilizer systems includes:
a cushioning magnetic system configured to move the mandible downward with respect to the maxilla of the user.

18. The mouthguard system of claim 13 wherein the cushioning magnetic system includes:
a first magnetic element coupled to the upper mouthguard assembly; and
a second magnetic element coupled to the lower mouthguard assembly.

19. A mouthguard system comprising:
an upper mouthguard assembly configured to be positionable about a maxilla of a user;
a lower mouthguard assembly configured to be positionable about a mandible of the user; and
one or more stabilizer systems configured to align the upper mouthguard assembly with the lower mouthguard assembly, wherein the one or more stabilizer systems includes:
a cushioning magnetic system configured to move the mandible downward with respect to the maxilla of the user and including:
a first magnetic element coupled to the upper mouthguard assembly, and
a second magnetic element coupled to the lower mouthguard assembly,
wherein the first and second magnetic elements are configured to generate a repelling magnetic force.

20. The mouthguard system of claim 1 wherein the one or more stabilizer systems includes:
a displacement magnetic system configured to move the mandible forward with respect to the maxilla of the user.
